(19) United States
(12) Patent Application Publication
(10) Pub. No.: US 2020/0147230 A1
(43) Pub. Date: May 14, 2020

(54) TUMOR NECROSIS TARGETING COMPOSITIONS AND METHODS

(71) Applicant: Cancer Therapeutics Laboratories, Inc., Los Angeles, CA (US)

(72) Inventor: Alan EPSTEIN, Los Angeles, CA (US)

(73) Assignee: Cancer Therapeutics Laboratories, Inc., Los Angeles, CA (US)

(21) Appl. No.: 16/496,173

(22) PCT Filed: Mar. 19, 2018
(86) PCT No.: PCT/US2018/023122

§ 371 Date: Sep. 20, 2019

Related U.S. Application Data
(60) Provisional application No. 62/473,552, filed on Mar. 20, 2017.

(51) Int. Cl. A61K 47/68; C07K 16/28; A61K 51/10; A61K 45/06

(52) U.S. Cl. CPC A61K 47/6801; C07K 16/28; A61K 2039/505; A61K 45/06; A61K 51/1027

(57) ABSTRACT

compositions and methods employ an anti-nucleolin antibody or fragment thereof to specifically target necrotic tumor cells. Thus, and in preferred aspects, compositions and methods may be used to target the tumor microenvironment and cancer cells within such milieu. Also included are methods of targeting necrotic cells with binding agent that specifically binds nucleolin.

### CROSS-REFERENCE TO RELATED APPLICATION

This application claims the benefit of priority from U.S. Provisional Patent Application Ser. No. 62/473,552, filed on Mar. 20, 2017, the contents of which are incorporated herein by reference.

### FIELD OF THE INVENTION

The field of the invention is compositions and methods for targeting necrotic cells, and especially necrotic cells in a solid tumor.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Targeting tumors has long been a desirable strategy for cancer treatment, and numerous approaches have been undertaken. For example, tumor cells can be targeted using one or more cancer associated antigens, cancer specific antigens, or tumor and patient specific neoepitopes. Such approaches advantageously enable delivery of various therapeutic moieties, however, tend to either lack specificity to cancer cells only, or have exclusive specificity to a single patient. In further known approaches, rapidly dividing tumor cells can also be targeted using molecules that are up-regulated during exponential growth. For example, cell surface nucleolin has been used as a potential target for anti-cancer therapies in which inhibitors were intended to interfere with the surface nucleolin of a variety of dividing cancer cells (e.g., using a pseudopeptide ligand as described in Cancer Res 2011; 71: 3296-305, BMC Cancer 2010; 10: 325, or PLoS One 2008; 3(6): e2518).

Unfortunately, many cancer cells will not rapidly divide in the frequently hypoxic tumor microenvironment and tend to undergo epithelial to mesenchymal transition, thereby leading to a population of difficult to treat cells and immune evasion. Tumor necrosis is a key characteristic of all cancers, particularly in the frequently hypoxic tumor microenvironment, and is typically not found in normal tissues and organs. Hence, targeting necrosis would provide site-specificity to targeting and would at least conceptually be applicable to all human tumors. In this context it

should be noted that targeting tumor necrosis should be viewed as a delivery method for various immunoactive molecules and drugs to the interior of tumors, and not as a method of directly killing tumor cells. It would therefore be desirable to have target molecules that are present and persistent in a tumor microenvironment to so allow directed therapy. However, to date no established therapeutic interventions are known that make use of such target molecules.

Therefore, there remains a need for compositions and methods to specifically target the tumor microenvironment.

### SUMMARY OF THE INVENTION

The inventive subject matter is directed to various compositions, systems, and methods of targeting necrotic cells, and especially human necrotic cells in a tumor microenvironment using an antibody that selectively binds to nucleolin. Indeed, the inventor has discovered that nucleolin is a common and persistent target in the tumor microenvironment, and especially in non-living and necrotic cells and cell fragments.

In one aspect of the inventive subject matter, the inventor contemplates a method of targeting a necrotic cell that includes a step of contacting the necrotic cell with a binding agent that specifically binds nucleolin. Most typically, the binding agent is an antibody, and antibody fragment, or an agent isolated from phage display or RNA display. Moreover, it is generally preferred that the necrotic cell is a tumor cell, which may be located in the tumor microenvironment. While not limiting to the inventive subject matter, it is generally contemplated that the nucleolin will be located within or on the surface of the necrotic cell, and/or that the step of contacting is performed in vivo.

In further contemplated aspects, the binding agent may also be coupled to a therapeutic agent and/or imaging agent. For example, suitable therapeutic agent include cytokines or portions thereof, chemokines or portions thereof, inhibitors of myeloid-derived suppressor cell (MDSC) or M2 macrophages, radioisotopes, co-stimulatory molecules, toll-like receptor (“TLR”) agonists and ligands, molecules interfering with epithelial mesenchymal transition (“EMT”), and various other known chemotherapeutic drugs, and suitable imaging agents include radioisotopes, positron emission tomography (PET) labels, and single-photon emission computed tomography (SPECT) labels.

Thus, and viewed from a different perspective, the inventor also contemplates a method of targeting a tumor microenvironment that contains necrotic cells. In such method, the necrotic cells (e.g., tumor cells) in the microenvironment are contacted with a binding agent (e.g., antibody, antibody fragment, agent isolated from phage display or RNA display) that specifically binds nucleolin. Most typically, the necrotic cell is a tumor cell in a solid tumor, the nucleolin is located within the necrotic cell, and/or the step of contacting is performed in vivo. As noted above, the binding agent may be coupled to a therapeutic agent and/or imaging agent. For example, suitable therapeutic agent includes cytokines or portions thereof, chemokines or portions thereof, inhibitors of MDSCs or M2 macrophages, radioisotopes, co-stimulatory molecules, TLR agonists and ligands, molecules interfering with EMT, and various other known chemotherapeutic drugs, and suitable imaging agents include radioisotopes, PET labels, and SPECT labels.

In yet another aspect of the inventive subject matter, the inventor further contemplates a method of delivering a therapeutic agent to a tumor microenvironment containing necrotic tumor cells. Most typically, such method will include a step of providing a therapeutic agent that is coupled to a binding agent that specifically binds nucleolin; and a further step of contacting the necrotic tumor cells in the microenvironment with the therapeutic agent under conditions that allow the binding agent to bind to nucleolin in the necrotic cell in the tumor microenvironment.

In further contemplated aspects, the therapeutic agent comprises at least one of a cytokine or portion thereof, a chemokine or portion thereof, an inhibitor of an MDSC, an inhibitor of an M2 macrophage, and a radioisotope, and/or preferred binding agents include an antibody, and antibody fragment, or an agent isolated from a phage display or RNA display. Moreover, it is generally preferred that the step of contacting is performed in vivo. In such case, contemplated methods may also include a step of administering a vasculature permeability enhancing agent.

Similarly, the inventor also contemplates a method of delivering an imaging agent to a tumor microenvironment containing necrotic tumor cells. Preferred methods will include a step of providing an imaging agent that is coupled to a binding agent that specifically binds nucleolin; and another step of contacting the necrotic tumor cells in the microenvironment with the imaging agent under conditions that allow the binding agent to bind to nucleolin in the necrotic cell in the tumor microenvironment.

As noted earlier, the imaging agent may comprise at least one of a radioisotope, a PET label, and a SPECT label, and/or the binding agent may be an antibody, and antibody fragment, or an agent isolated from a phage display or RNA display. In addition, it is contemplated that such methods may further comprise a step of administering a vasculature permeability enhancing agent.

Consequently, the inventors also contemplate a therapeutic hybrid molecule that comprises a binding agent that specifically binds nucleolin, wherein the binding agent is coupled to a therapeutic agent, and contemplate a diagnostic hybrid molecule comprising a binding agent that specifically binds nucleolin, wherein the binding agent is coupled to an imaging agent. With respect to the binding agent, the therapeutic agent, and the imaging agent, the same considerations as noted above apply. Moreover, such hybrid molecules may be formulated into a pharmaceutical composition for administration to a mammal (and especially human) diagnosed with a tumor or necrotic tissue.

Thus, use of a binding agent that specifically binds nucleolin to target a necrotic cell, and use of a binding agent that specifically binds nucleolin to target a necrotic tumor cell in a tumor microenvironment are especially contemplated. Similarly, use of a binding agent that specifically binds nucleolin for targeted delivery of a therapeutic agent or an imaging agent to a necrotic cell in a tumor microenvironment is contemplated.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is an exemplary SDS-PAGE with nucleolin captured from immunoprecipitation using NANT-1, and the human nucleolin sequence (SEQ ID NO:1) with various protein fragments identified from the captured nucleolin.

FIGS. 2A, 2B and 2C are photomicrographs of colon 26 cells (FIG. 2A) and Raji cells (FIGS. 2B and 2C) stained with secondary antibodies against NANT-1.

FIGS. 3A-3C depict graphs from fixed cell assays using NANT-1 (364-5-10-5) and control antibodies in the context of specific cells.

FIG. 4 is a graph from a HEY ghost cell assay NANT-1 (364-5-10-5) and control antibodies.

FIGS. 5A-5B depict graphs from exemplary uptake experiments.

FIGS. 6A-6D depict graphs from exemplary comparative biodistribution experiments of radioiodinated NANT-1 at 0.1 mg/kg vs. 1 mg/kg.

### DETAILED DESCRIPTION

The inventive subject matter is directed to the discovery that nucleolin is a highly specific target for necrotic cells, and especially necrotic tumor cells, which is particularly unexpected as nucleolin is typically associated with rapidly dividing cells and as nucleolin is also often quickly degraded to fragments in resting or non-rapidly dividing cells.

Based on the unexpected discovery that nucleolin is a commonly available and persistent target in necrotic cells and cell fragments, and especially in a tumor microenvironment, it is now contemplated that the tumor microenvironment can be effectively addressed with various agents that may be used for diagnosis and/or therapy. Notably, so detected nucleolin in necrotic cells is preferentially located in the nuclear and perinuclear compartments, and to a significantly lesser degree (or below detection limit) on the cell membrane of necrotic cancer cells. Consequently, as the tumor microenvironment frequently presents a difficult-to-target environment that promotes various mechanisms of immune evasion (e.g., hypoxia reducing activity of NK cells, lack of nutrients and oxygen promoting EMT, etc.), specific delivery and retention of various immune stimulating factors is thought to particularly benefit immune therapy.

In that regard, it should be appreciated that the terms ‘apoptosis’ and ‘necrosis’ are not interchangeably used herein, but refer to two principally distinct mechanisms and pathways of cell death. While apoptosis is a well-defined process of programmed cell death involving specialized signaling events and staged cell shut-down (e.g., blebbing, cell shrinkage, nuclear fragmentation, chromatin condensation, DNA fragmentation, mRNA decay), necrosis is typically evidenced as a disorganized process of cell death with concomitant loss of organelle function, cell rupture, and release of cell content into the environment. Furthermore, necrosis is typically accompanied by an inflammatory cell response. Moreover, it should be appreciated that necrosis is the site by which the immune system “sees” the tumor and reacts immunologically. This is important since delivering payloads to necrosis will aid the immune system in recognizing and reacting to a tumor, and thus is the preferred site of delivery of these therapeutically effective payloads.

Based on the inventor's discovery as described in more detail herein, the inventor thus contemplates the use of antibodies (and fragments thereof) and other specific binding agents such as RNA display selected proteins or phage display selected proteins to deliver an imaging and/or therapeutic agent to the tumor microenvironment. Most typically, the antibody, or fragment thereof or other binding agent will have specific binding (i.e., binds with a Kd of less than 10⁻⁷ M, and more typically less than 10⁻⁸ M as, for example, determined by SPR or other technique) to human nucleolin. For example, there are many commercially available monoclonal and polyclonal anti-nucleolin antibodies known in the art (e.g., Abcam ab136649, Millipore MABC587, clone 364-5-10-5), and all of those are deemed suitable for use herein. However, preferred antibodies include human anti-nucleolin antibodies and humanized anti-nucleolin antibodies, preferably of IgG subtype. In addition, it is generally preferred that the antibody has specificity towards human nucleolin.

Similarly, all fragments of antibodies are also contemplated so long as such fragments still retain binding specificity for nucleolin (and most typically human nucleolin). Thus, suitable antibody fragments include scFv (single chain variable fragment), Fab-type antibodies, sdAb (single domain antibodies), and chimeric antibodies with at least a second protein domain that will provide one or more additional functions. Likewise, where the binding domain is an artificially selected domain (e.g., via RNA or phage display), Fc and other fusion proteins containing such artificially selected domains are also deemed appropriate.

In further contemplated aspects, it should be noted that bi- and multi-specific antibodies and binding molecules are also deemed suitable for use herein where at least one binding domain has specific binding for nucleolin (i.e., binds with a Kd of less than 10⁻⁷ M, and more typically less than 10⁻⁸ M). For example, suitable bi- and multi-specific antibodies include bispecific Fab₂, bispecific diabodies, trispecific Fab₃, and trispecific triabodies.

Regardless of the type of antibody or binding molecule, it is generally contemplated that the antibody or binding molecule will be coupled to a diagnostic and/or therapeutic agent. Most typically, the coupling will be covalent coupling, which may be achieved using conventional coupling chemistry such as amino group reactive reagents (e.g., N-hydroxysuccinimide esters, various aldehydes, carbodiimide compounds, epoxides, imidoesters, etc.), or sulfhydryl group reactive reagents (e.g., various maleimides, thiols, etc.), or may be implemented via recombinant cloning techniques in which the antibody (fragment) is fused in frame to an optional linker that is fused in frame to the second protein of interest. Suitable linkers may be selected by a desired length (e.g., to provide a desired spatial distance), amino acid composition (e.g., to provide a cleavable linker or flexible linker), etc. In still further contemplated modes of coupling, coupling may be non-covalently and in especially preferred manners, the coupling is provided by elements of known binding pairs, such as biotin/avidin, cellulose/cellulose binding protein, nickel-nitrilotriacetic acid (Ni-NTA)/oligo-histidyl, etc.

With respect to diagnostic agents, it is contemplated that all detectable (and preferably quantitatively detectable) agents are deemed suitable for use herein. Furthermore, it should be noted that the detection may be performed ex vivo (e.g., on tissue section) and/or in vivo using suitable methods known in the art. For example, visually detectable imaging agents include fluorophores, luminescent groups, catalytically active groups (e.g., to precipitate a dye and/or activate a chromogen or luminogen), radiographically detectable groups (e.g., PET, SPECT, NMR label, radioisotope, etc.).

Likewise, and with respect to therapeutic agents, it is contemplated that all therapeutic agents are deemed appropriate for use herein. However, in particularly preferred aspects of the inventive subject matter, the therapeutic agent will have an immune stimulatory effect. Most typically, such stimulator effect will reverse or neutralize one or more mechanisms that lead to immune evasion of cancer cells in the tumor microenvironment. For example, where the immune evasion is based on the recruitment of M2 macrophages or regulatory T-cells (Tregs), suitable therapeutic agents will include those that specifically deactivate or destroy such inhibitory cells (e.g., gemcitabine, RP-182 (see SEQ ID NO: 121 of U.S. Pat. No. 9,492,499), or cyclophosphamide). Additionally, or alternatively, where the immune evasion is based on checkpoint inhibition with effector and/or helper cells, binders or antagonists to CTLA4 or PD1 (e.g., ipilimumab, pembrolizumab, etc.) are contemplated.

Conversely, it should also be appreciated that an immune therapy may be enhanced by use of a therapeutic agent where the therapeutic agent has immune stimulatory activity. Such immune stimulatory activity can be achieved via use of co-stimulatory signals that are coupled to the nucleolin binder, preferably in the context of one or more tumor (neo)antigens. For example, co-stimulatory signals include 4-1BBL, OX40L, GITRL, TIM3, LFA3, ICAM1, ICOSL, etc. In addition, it should be appreciated that immune stimulatory agents will also include immune stimulating cytokines such as IL-2, IL-12, IL15, IL-15 superagonists, TLR agonists and ligands, etc. Still further, it should be appreciated that the therapeutic agent may also comprise a (pro-inflammatory) chemokine that will attract further immune competent cells.

Where desired, the therapeutic agent may also include agents that will target factors that contribute to EMT (epithelial mesenchymal transition) in the tumor microenvironment, including IL-8 and TNF-β. Therefore, suitable therapeutic agents will also include those that bind or otherwise sequester IL-8 and TNF-β.

Additionally, the therapeutic agent may also include more conventional drugs used in the treatment of cancer. For example, typical anticancer drugs include antimetabolites, drugs that interfere with microtubule formation or disassembly, DNA alkylating agents, and topoisomerase inhibitors, cytotoxic drugs, etc., all of which may be cleavable under conditions prevalent in the tumor microenvironment. Contemplated therapeutic agents also include radiotherapeutic agents such as alpha and beta emitters (e.g., Bi-213, Pb-212, 1-131, Ac-225, Sr-89, etc.).

Consequently, and as is shown in more detail below, the inventors generally contemplate a method of targeting a necrotic cell (typically a tumor cell, most typically a necrotic tumor cell in a tumor microenvironment) that includes a step of contacting the necrotic cell with a binding agent that specifically binds nucleolin. As noted above such binding agent is most typically an antibody, an antibody fragment, or an agent selected from phage or RNA display. Moreover, the contacting can be performed in such methods in vivo or in vitro. For example, where the step is performed in vitro, relatively small quantities (e.g., between 0.001-100 μg, or between 0.01-0.1 μg, or between 0.001-0.01 μg) of the binding agent may be required. On the other hand, where the step is performed in vivo, relatively large quantities (e.g., between 0.01-100 mg, or between 0.1-10 mg, or between 1-10 mg) of the binding agent may be required. Of course, it should be appreciated that where the binding agent is coupled to an imaging and/or therapeutic agent, the quantity of the binding agent will also be at least in part determined by the type and quantity of the imaging and/or therapeutic agent needed for the desired effect.

Consequently, the inventor also contemplates a method of delivering a therapeutic and/or imaging agent to a tumor microenvironment containing necrotic tumor cells. As noted above, such method will typically include a step of providing a therapeutic agent that is coupled to a binding agent that specifically binds nucleolin, and a further step of contacting (preferably in vivo) the necrotic tumor cells in the microenvironment with the therapeutic agent under conditions that allow the binding agent to bind to nucleolin in the necrotic cell in the tumor microenvironment.

In addition, the methods contemplated herein may further include one or more steps of increasing tumor necrosis to thereby enhance uptake of the modified antibody or binder into the tumor to so optimize the delivery of a therapeutic or diagnostic payload. For example, suitable further steps include radiotherapy, chemotherapy, or immunotherapy, and especially low-dose metronomic chemotherapy and radiotherapy.

### Examples

Sequencing:

The NANT-1 antibody sequence information was derived from the mRNA of hybridoma cells (murine) producing NANT-1 following standard protocols well known in the art. As the sequences for murine IgG₁ isotype is known, only variable heavy and light chain information is provided below, with respective CDR regions underlined:

Heavy Chain Variable Region Sequence (SEQ ID NO:2):

QESGPQLVRPGASVKISCKASGYSFTSYWMHWVKQRPGQGLEWIGMIDPS
DSETRLNQKFKDKATLTVDKSSSTAYMQLNSPTSEDSAVYYCARDGGYYA
WFAYWGQGTLVTVSA

Light Chain Variable Region Sequence (SEQ ID NO:3):

DIVLTQTPKSMSMSVGERVTLTCKASENVVTYVSWYQQKPEQSPKLLIYG
ASNRYTGVPDRFTGSGSATDFTLTISSVQAEDLADYHCGQGYSYPYTFGG
GTKLEIKRA

Immunoprecipitation and Mass Spectroscopy:

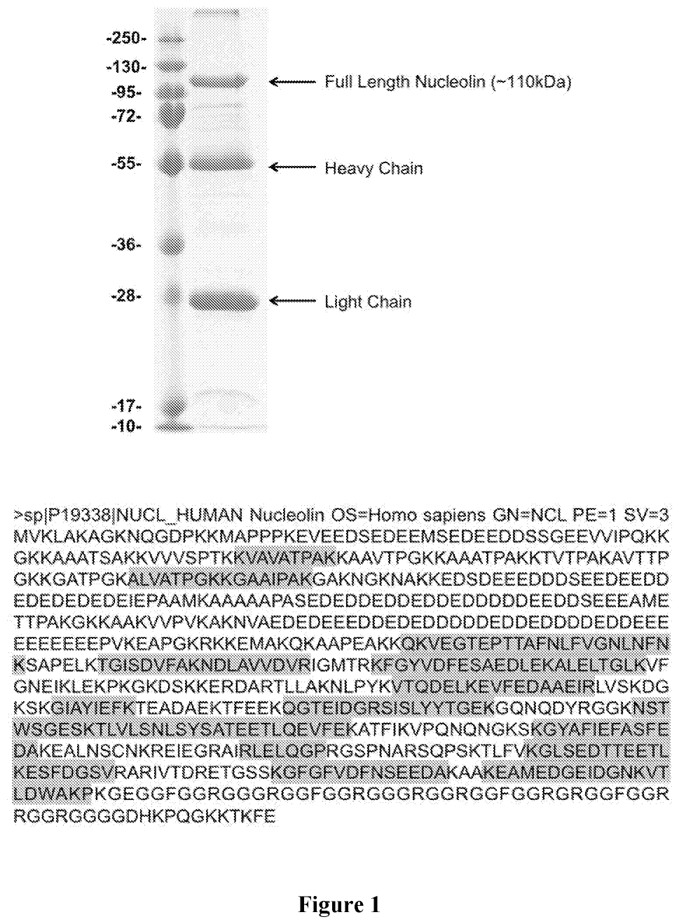

The NANT-1 antibody was subjected to an immunoprecipitation assay after conjugation to Protein-A Sepharose beads in order to confirm its binding to human nucleolin. For these studies, cold lysates of the human colon carcinoma cell line HT29 prepared in RIPA buffer were obtained and incubated with NANT-1 protein-A beads overnight at 4° C. using continuous rotation. After incubation, the beads were washed 3× in phosphate buffered saline (PBS) and then 3× in 0.5M LiCl to remove unbound proteins. The washed beads were then subjected to SDS PAGE electrophoresis and stained briefly with Coomassie Blue to detect the separated proteins. The faint band at 110 Kd as can be seen in FIG. 1 (arrow) was then excised and submitted to mass spectroscopy to confirm its identity as nucleolin. More specifically, human colon cancer HT29 cells were lysed and sonicated in radioimmunoprecipitation assay buffer (RIPA) buffer. Lysate was then incubated overnight with Protein A beads bound to NANT-1. After washing, the antibody and its antigen were eluted with 50 Mm Glycine (pH 2.5) and neutralized with 10% 1.5M Tris-HCl (pH 8.8). As shown in the left panel of FIG. 1, the molecular weights of the eluent bands were analyzed with SDS-Page Ruler Plus Prestained Protein Ladder in a 12% Tris-Glycine Polyacrylamide Gel. After Coomassie blue staining, the faint band at −110 kDa was extracted and sent for LC-MS which confirmed the presence of human nucleolin with >99.8% initial probability at 35.2% sequence coverage. As shown in the right panel, the highlighted areas show sequences detected by mass spectroscopy that are identical to human nucleolin.

Indirect Immunofluorescence:

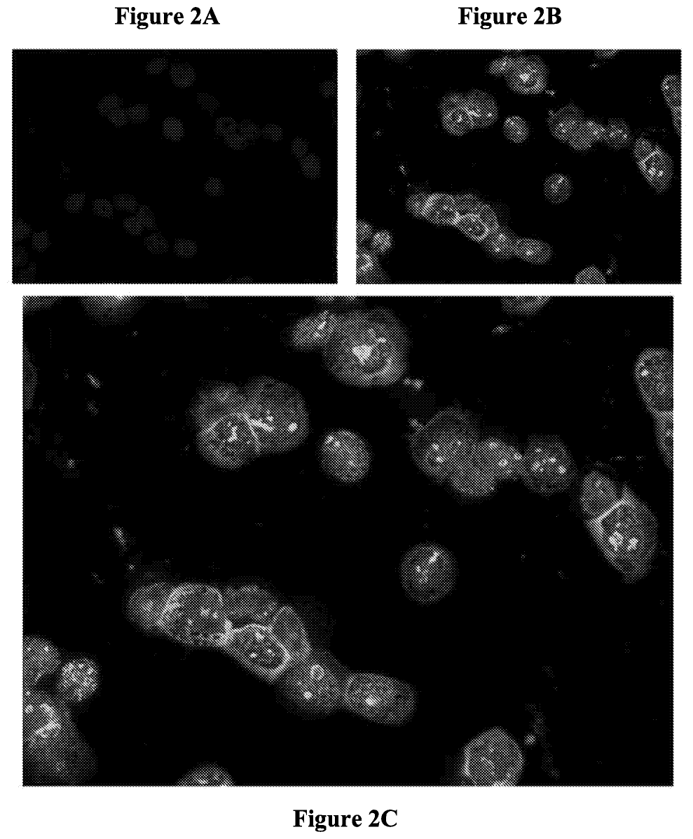

To demonstrate the localization of NANT-1 in fixed cell preparations and determine its specificity to human cells, indirect immunofluorescence assays were performed on a number of human and mouse cell lines, and exemplary results are shown in FIGS. 2A, 2B and 2C, below. For the tested cell lines, the data demonstrate that NANT-1 localized to the nucleolus and peri-nuclear cytoplasm of human cells, but exhibited no binding to tumor cells of murine origin. For these procedures, human and mouse cell lines were air dried onto printed microscope slides, fixed with 2% paraformaldehyde (EM grade) at room temperature for 10 minutes, permeabilized with 1% Triton X-100 in PBS briefly, and blocked with 5% BSA for 1 hour at 25° C. Samples were then incubated with the primary NANT-1 antibody (25 ug/mL) for 1 hour. After washing the slides with PBS to remove unbound antibody, the wells were then incubated for 1 hr at room temperature with FITC-conjugated goat anti-mouse F(ab)₂ (1/2,500) from Jackson Immunology as the secondary antibody. As the final step, the slides were incubated briefly with 4′,6-diamidino-2-phenylindole (DAPI) (blue fluorescence) to counterstain the nuclei and were observed and photographed using a Leitz Orthoplan immunofluorescence microscope using a water immersion 50× objective and photographed using an Immunofluorescence confocal microscope.

As is evident from FIG. 2A, Colon 26 murine colon carcinoma cells showed no binding of NANT-1 by these methods. By contrast, as can be seen in FIGS. 2B and 2C, human Burkitt's lymphoma Raji cells showed clear localization of antibody in the nucleolus and peri-nuclear cytoplasm (green fluorescence). Cells were counterstained with DAPI which stains the nuclei blue (×50 water objective).

Fixed Cell Assay:

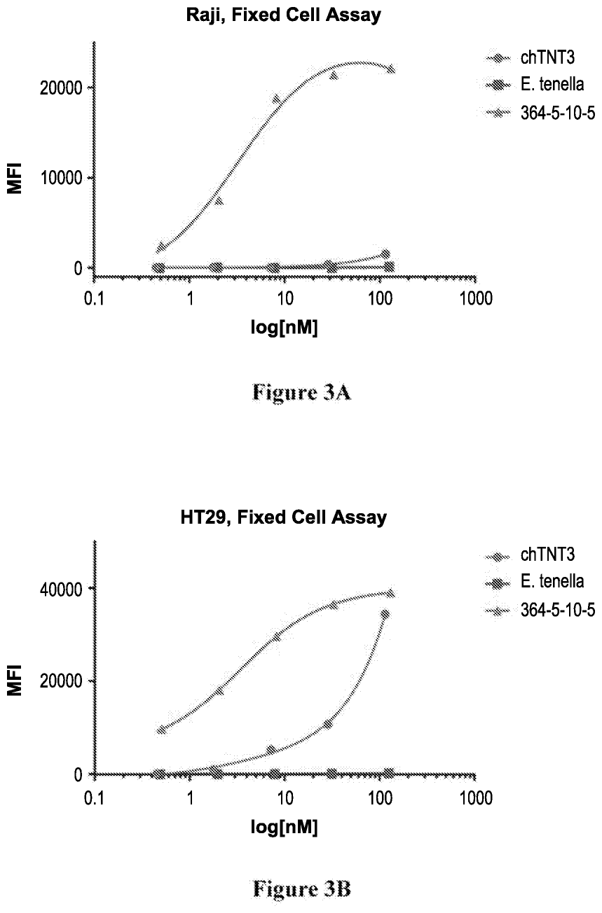

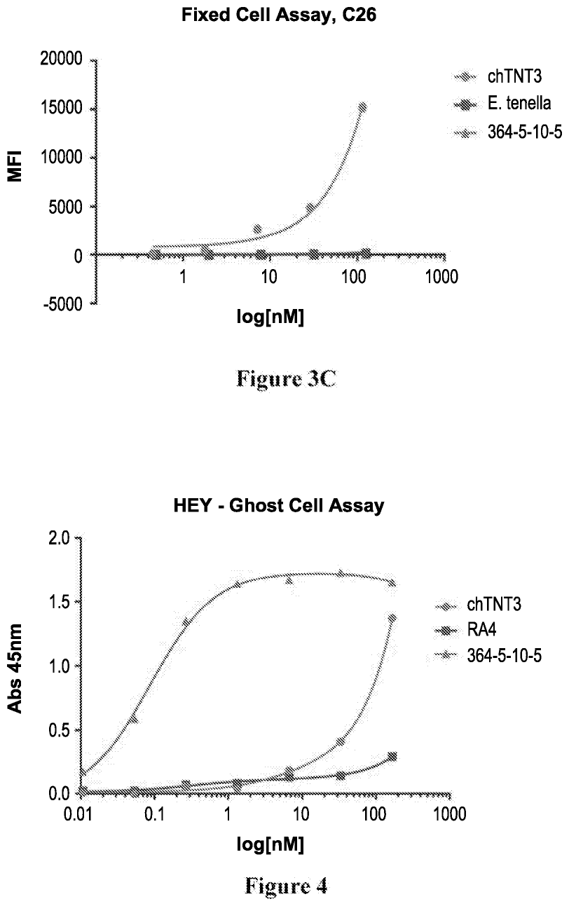

Human tumor cell lines Raji and HT29 and the murine tumor cell line C26 were fixed with 2% paraformaldehyde (EM Grade, Polysciences) for 15 min and then permeabilized with 0.5% Triton X-100 in PBS for 10 minutes at room temperature. Samples prepared in triplicate were then incubated with the primary antibody NANT-1 (25 ug/mL) for 1 hour. After a PBS rinse, the cells were then stained with the secondary antibody FITC-conjugated anti-mouse F(ab)₂ (1/2500; Jackson Immunology) for 1 hr and then washed with PBS. Fluorescence was measured in a BioTek Synergy HT spectrophotometer to determine the MFI. The data were plotted as shown in FIG. 3A-3C to obtain binding data used to calculate Kd and R² against each fixed cell line as shown in Table 1 below. In these studies, an IgG1 isotype control antibody was used as a negative control.

In the experiments of FIGS. 3A-3C, an In Vitro fixed assay demonstrating binding of NANT-1 to Human Nucleolin by fluorescence-activated cell sorting (FACS) Analysis is shown. Nuclei were counter stained with DAPI (blue fluorescence). The results demonstrate binding of NANT-1 to human Raji and HT-29 cells but not to the murine colon carcinoma cell line C26 indicating the human specificity of the antibody. By contrast, chTNT-3 bound to both human and mouse cells by these assays as expected. In comparison, the NANT-1 antibody showed a higher avidity for HT-29 fixed cells than chTNT-3.

TABLE 1

| | chTNT3 | Isotype | |
|---|---|---|---|
| Raji | (+control) | Control | NANT-1 |
| Kd(nM) | ~377119 | ~1300 | 3.340 |
| R² | 0.9971 | 0.9046 | 0.9871 |
| | | Isotype | |
| HT29 | chTNT3 | Control | 364-5-10-5 |
| Kd(nM) | 2.598 | ~0.0002408 | 3.450 |
| R² | 0.9987 | 0.9970 | 1.000 |
| | | Isotype | |
| C26 | chTNT3 | Control | 364-5-10-5 |
| Kd(nM) | ~9.335e+03 | 33316 | ~126509 |
| R² | 0.9867 | N/A | 0.9796 |

In Vitro Ghost Cell Assay:

To assess further the targeting of NANT-1 to necrosis, an additional in vitro assay, the Ghost Cell Assay, developed in our laboratory was performed using HEY human ovarian carcinoma cells. For this assay, HEY cells were grown as monolayers in triple flasks using RPMI-1640 medium containing 8% FCS and 1% antibiotics solution. Once confluent, the cells were trypsinized, washed, and resuspended in 10 ml of PBS, before being subjected to three cycles of freeze/thawing using liquid nitrogen and a 37° C. water bath. After each cycle of thawing, the cells were washed with 50 ml of PBS and pelleted by centrifugation at 1,000 rpm. After completion of the three cycles, the cell ghosts were resuspended in 10 ml PBS and 100 ul were then added in triplicate to a 96 well microtiter plate. The cell ghosts were then washed 4× in PBS containing 0.05% Tween-20 and then blocked for 2 hr with 300 ul using the same diluent at room temperature using continuous shaking. After blocking, 10-fold dilutions starting at 2 ug/ml of freshly biotinylated 364-5-10-5 is added in triplicate and incubated for 2 hr at room temperature with continuous shaking. After washing with diluent 4×, the secondary reagent of streptavidin-HRPT is added (1 in 5,000 dilution; Jackson Immunoresearch) for an additional 1 hr of incubation at room temperature with shaking. After an additional wash to remove unbound streptavidin, the TMB substrate (Biolegend) is added the plates were read in a BioTek Synergy HT spectrophotometer at 450 nm.

The data for this experiment are shown in FIG. 4 and below by Table 2 which show an excellent binding curve for NANT-1 and antibody affinity to cell ghosts. By comparison, the negative control antibody RA4 to human CD25 showed little binding and chTNT-3 had a much lower affinity for the ghost cell preparation. The ghost cell assay provides a means of identifying if the antigen of interest is retained after cell lysis when soluble cellular components are lost to the environment. It is a more stringent test for targeting necrotic regions of tumors or tissues than the fixed cell assay shown above.

TABLE 2

| Metric | chTNT3 | RA4 | NANT-1 |
|---|---|---|---|
| Kd(nM) | 5.121 | 0.3080 | 0.08661 |
| R square | 0.9985 | 0.9869 | 0.9971 |

In Vivo Biodistribution Studies:

In order to demonstrate the specific targeting of NANT-1 to human tumors heterotransplanted in nude mice, tissue biodistribution studies were performed. For these studies, 6-8 week old female athymic nude mice (Jackson Laboratories, Inc.) were implanted with 5×106 HT-29 human colon carcinoma cells in the left flank using a 0.2 ml inoculum and 25 gauge needle. The tumors were grown until they reached approximately 1 cm in diameter (7-10 days). Within each group (n=5), individual mice were injected i.v. with a 0.1 ml inoculum containing 100 μCi/10 μg of 125 I-labeled MAb. Mice were sacrificed at various times post-injection, and organs, blood, and tumors were removed and weighed. The radioactivity in the samples was then measured and expressed as % ID/g and tumor/organ ratios (cpm per gram tumor/cpm per gram organ). Significance levels were determined using the Wilcox rank sum test.

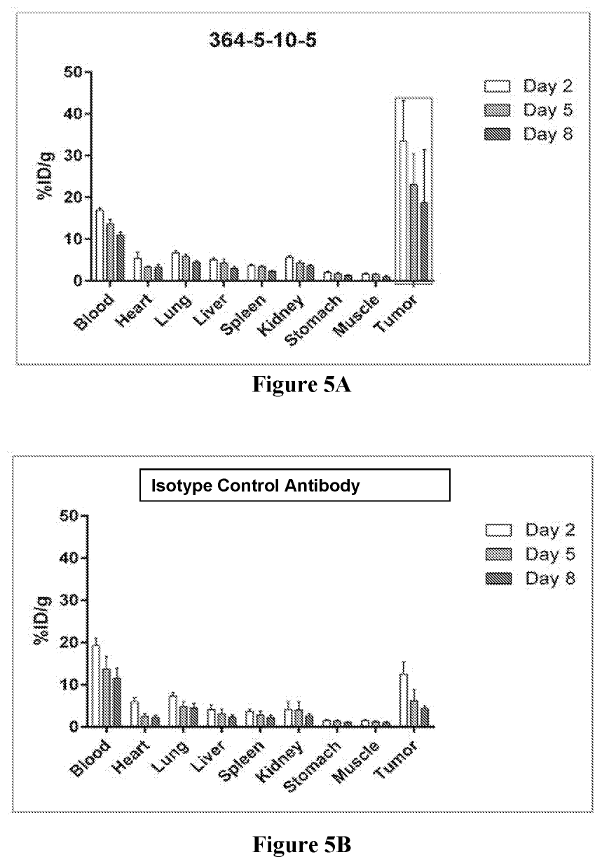

As shown in FIGS. 5A-5B, antibody NANT-1 showed excellent and specific uptake in the HT-29 tumor compared to the isotype control antibody. Here, the results illustrate comparative biodistribution studies of (5A) NANT-1 (murine antibody 364-5-10-5) and (5B) isotype control antibody in HT29 xenograft nude mouse model at 2, 5. and 8 days. For these studies antibodies were radiolabeled with 1251 and injected iv in groups of tumor bearing nude mice (n=5). Tumor and tissues were then removed at autopsy at the days shown to quantitate antibody uptake in each sample. Uptake levels were in excess of 30% injected dose/gram at day two and decreased to approximately 20% at days 5 and 8. By contrast, uptake in blood was around 18% at day 2 and decreased to around 10% by day 8. Normal organs had insignificant uptake which decreased by day 8 to less than 2-4% depending on the organ as the blood pool cleared from each tissue. For the isotype control set of mice, no uptake was seen other than blood pool in all tissues and tumor.

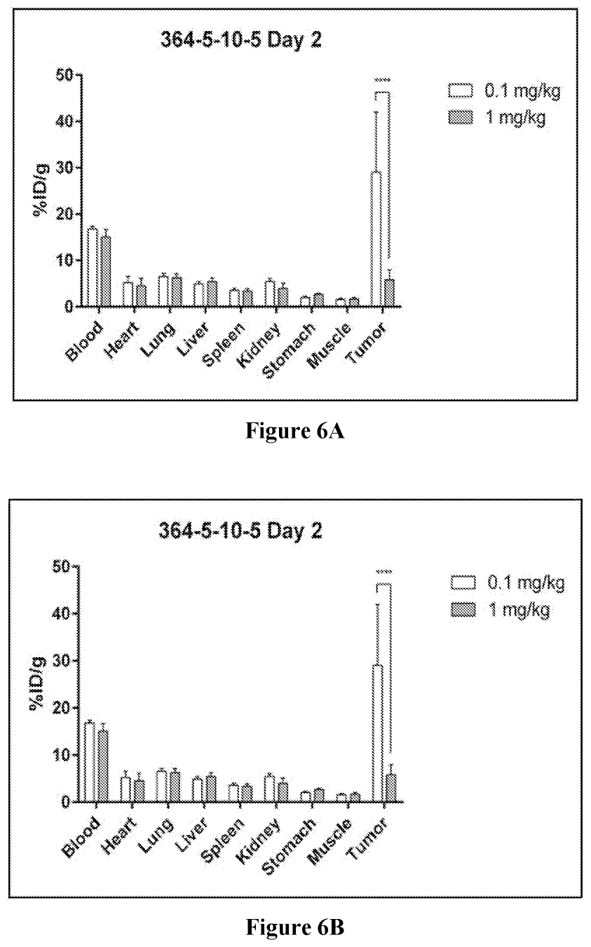

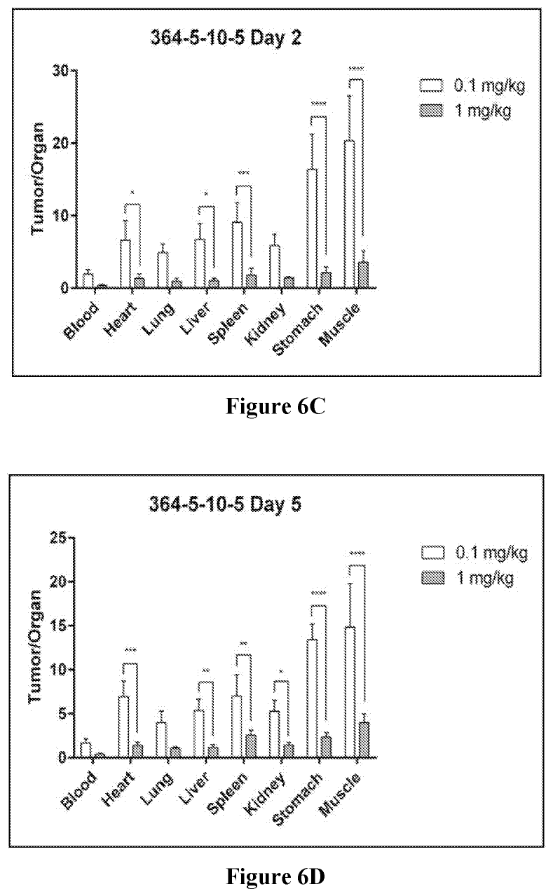

In FIGS. 6A-6D, comparative biodistribution analyses of 0.1 (radiotracer dose) vs. 1 mg (radiotracer+cold dose) of radiolabeled 364-5-10-5 were performed to confirm antigen specificity in the tumor. As such, it is practical to employ radiotracer studies using radiolabeled antibody at a fixed radiotracer dose vs. an increasing concentration of the respective cold antibody which is designed to reveal highly specific tumor uptake and high expression level (i.e. higher tumor/organ ratio at a given time post-injection). It is important to bear in mind that, as the dose of unlabeled antibody is increased, increasing antigen-receptor occupancy levels means that the radioactivity levels in tissues actually decrease based on the concept of competitive binding inhibition. In this situation, the radiotracer is used as a marker to follow the antibody levels in the tumor. At a fixed dose of radiotracer, radioactivity levels in the tumor decrease with increasing dose of unlabeled antibody due to competitive binding, reaching a lower uptake at higher antigen-receptor occupancy.

More particularly, FIGS. 6A-6D exemplarily illustrate comparative biodistribution data of radioiodinated NANT-1 at 0.1 mg/kg vs. 1 mg/kg and demonstrate nucleolin specificity of the antibody. Here, mice injected with 1 mg/kg (radiotracer+unlabeled antibody) of NANT-1 had a reduced tumor uptake than 0.1 mg/kg (radiotracer dose) at all time points compared to normal tissues where radioactivity uptake was similar. As shown in FIGS. 6A and 6B, the inverse relationship observed between NANT-1 dose and tumor uptake of radiolabeled antibody confirms the antigen specificity in tumor (which is not seen in normal tissues). The high tumor uptake (>20% ID/gm) and the tumor/organ ratio seen at the tracer dose, and at all time points, also confirms the high expression level of the target antigen in malignant vs. non-malignant tissues.

The exemplary data and further contemplations presented herein provide many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Moreover, as further used herein, and unless the context dictates otherwise, the term “coupled to” is intended to include both direct coupling (in which two elements that are coupled to each other contact each other), and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A method of targeting a necrotic cell, comprising contacting the necrotic cell with a binding agent that specifically binds nucleolin.

2. The method of claim 1 wherein the binding agent is an antibody, and antibody fragment, or an agent from a phage display or RNA display.

3. The method of claim 1 wherein the necrotic cell is a tumor cell.

4. The method of claim 1 wherein the necrotic cell is a tumor cell in a tumor microenvironment.

5. The method of claim 1 wherein the nucleolin is located within the necrotic cell.

6. The method of claim 1 wherein the step of contacting is performed in vivo.

7. The method of claim 1 wherein the binding agent further comprises at least one of a therapeutic agent and an imaging agent.

8. The method of claim 7 wherein the therapeutic agent comprises at least one of a cytokine or portion thereof, a chemokine or portion thereof, an inhibitor of an myeloid-derived suppressor cell (MDSC), an inhibitor of an M2 macrophage, a radioisotope, a co-stimulatory molecule, a toll-like receptor (“TLR”) TLR agonist or ligand, a molecule interfering with epithelial mesenchymal transition (“EMT”), and a chemotherapeutic drug.

9. The method of claim 7 wherein the imaging agent comprises at least one of a radioisotope, a PET label, and a SPECT label.

10-18. (canceled)

19. A method of delivering a therapeutic agent or an imaging agent to a tumor microenvironment containing necrotic tumor cells, comprising:
providing a therapeutic agent or an imaging agent that is coupled to a binding agent that specifically binds nucleolin; and
contacting the necrotic tumor cells in the microenvironment with the therapeutic agent or the imaging agent under conditions that allow the binding agent to bind to nucleolin in the necrotic cell in the tumor microenvironment.

20. The method of claim 19 wherein the therapeutic agent comprises at least one of a cytokine or portion thereof, a chemokine or portion thereof, an inhibitor of an myeloid-derived suppressor cell (MDSC), an inhibitor of an M2 macrophage, a radioisotope, a co-stimulatory molecule, a toll-like receptor (“TLR”) agonist or ligand, a molecule interfering with epithelial mesenchymal transition (“EMT”), and a chemotherapeutic drug.

21. The method of claim 19 wherein the tumor is a solid tumor.

22. The method of claim 19 wherein the binding agent is an antibody, and antibody fragment, or an agent from a phage display or RNA display.

23. The method of claim 19 wherein the step of contacting is performed in vivo.

24. The method of claim 19 further comprising a step of administering a vasculature permeability enhancing agent.

25. The method of claim 19 wherein the nucleolin in the necrotic cell is in an intracellular location.

26. (canceled)

27. The method of claim 19 wherein the imaging agent comprises at least one of a radioisotope, a PET label, and a SPECT label.

28-32. (canceled)

33. A hybrid molecule comprising a binding agent that specifically binds nucleolin, wherein the binding agent is coupled to a therapeutic agent or a diagnostic agent.

34. The hybrid molecule of claim 33 wherein the binding agent is an antibody, and antibody fragment, or an agent from a phage display or RNA display.

35. The hybrid molecule of claim 33 wherein the therapeutic agent comprises at least one of a cytokine or portion thereof, a chemokine or portion thereof, an inhibitor of an myeloid-derived suppressor cell (MDSC), an inhibitor of an M2 macrophage, a radioisotope, a co-stimulatory molecule, a toll-like receptor (“TLR”) agonist or ligand, a molecule interfering with epithelial mesenchymal transition (“EMT”), and a chemotherapeutic drug.

37-63. (canceled)


SEQUENCE LISTING (3 sequences)

SEQ ID NO: 1 - protein, 710 aa, Homo sapiens
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 2 - protein, 115 aa, Mus musculus
QESGPQLVRPGASVKISCKASGYSFTSYWMHWVKQRPGQGLEWIGMIDPSDSETRLNQKFKDKATLTVDKSSSTAYMQLNSPTSEDSAVYYCARDGGYYAWFAYWGQGTLVTVSA

SEQ ID NO: 3 - protein, 109 aa, Mus musculus
DIVLTQTPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRA